(19) United States
(12) Patent Application Publication
(10) Pub. No.: US 2010/0167387 A1
(43) Pub. Date: Jul. 1, 2010

(54) SYSTEM AND AUTOMATED DEVICE FOR ANALYSING A CELL SUSPENSION

(75) Inventor: Eric Peltier, Clamart (FR)

Correspondence Address: YOUNG & THOMPSON, 209 Madison Street, Suite 500, Alexandria, VA, 22314, US

(73) Assignee: NOVACYT, Velizy Villacoublay (FR)

(21) Appl. No.: 12/376,977

(22) PCT Filed: Aug. 1, 2007
(86) PCT No.: PCT/FR07/01329

§ 371 Date: Dec. 3, 2009

(30) Foreign Application Priority Data
Aug. 11, 2006 (FR) 06 07311

(51) Int. Cl. C12M 1/34

(52) U.S. Cl. 435/288.7; 435/287.1

(57) ABSTRACT

A system for analyzing a cell suspension includes at least one well for decanting (1) the suspension and elements (2, 3) for analyzing the cell deposits at the bottom of the well over time during the decanting, so as to allow an analysis of the cells by strata (T1, T2, T3) as a function of their rate of decantation.

The present invention relates to a system for analysing a cell suspension and an automated analysis device comprising such a system.

The analysis of a cell suspension and, for example, a fixed cytological smear is an extremely delicate operation.

The object of such an analysis is to screen pathological cells.

Various analysis techniques have already been proposed in the prior art.

These analyses may, for example, involve colouring operations, the object of which is to provide a colour and density contrast in the region of the nuclei and the cells.

However, this colouring operation is by no means specific and does not indicate tumorous cells in a specific manner. The identification of dysplastic or tumorous cells is carried out based on the appearances of the nuclei, and particularly their size, their shape, their density, etc., and on the nucleocytoplasmic ratio. An additional difficulty is linked to the low number of pathological cases (less than 5%) and the low number of pathological cells per case (less than 0.2%) which are sometimes masked by cell debris or inflammation.

Consequently, the work of a cytologist trained to select pathological cases is extremely demanding, unrewarding and a source of tension.

Solutions have also been proposed to improve this situation. One of these solutions is, for example, depositing thin layers of cells.

These deposits can be carried out, for example, by decanting a cell suspension, such as a cytological suspension, in order to allow these cells to be deposited on an analysis plate.

This analysis plate is then examined in order to detect the pathological cells.

However, these methods also have a given number of disadvantages, since the pathological cells can be relatively difficult to detect amongst the other deposits obtained.

The object of the invention is therefore to overcome these problems.

To this end, the invention relates to a system for analysing a cell suspension, characterised in that it comprises at least one pit for decanting the suspension and means for analysing the cell deposits at the bottom of the pit over time and during the decantation, in order to allow an analysis of the cells by means of strata in accordance with their decantation speed.

According to other features of the invention:
the analysis means comprise means for illuminating the bottom of the pit and means for photographing the deposits;
the pit has an open end which can be blocked by opaque means;
the opaque means are formed by means of a self-curing membrane; and
the cell suspension is a cytological suspension.

The invention also relates to an automated device for analysing a cell suspension comprising an analysis system according to the invention, means for taking a sample of cell suspension from a receptacle and means for pouring this sample into a decantation pit of the analysis system.

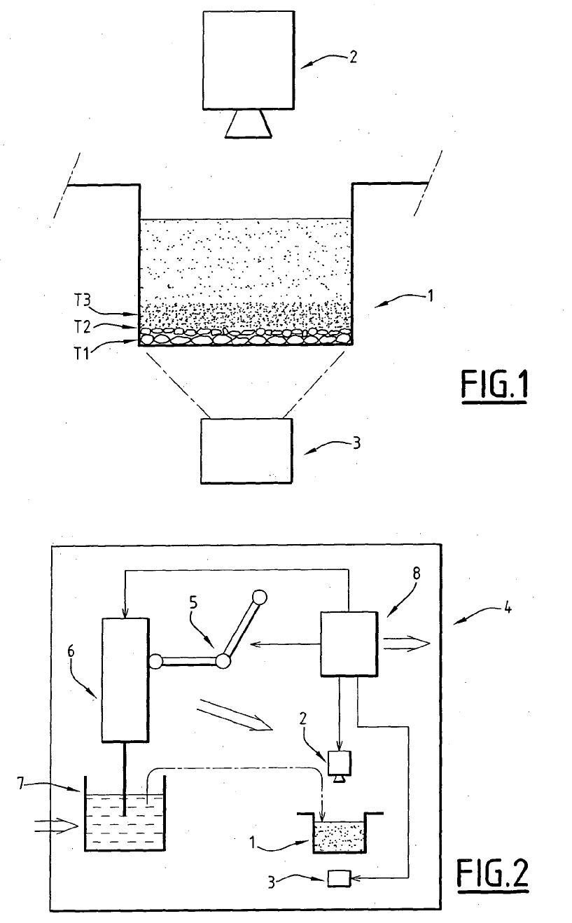

The invention will be better understood from a reading of the following description, given purely by way of example and with reference to the appended drawings, in which:
FIG. 1 is a schematic section of an analysis system according to the invention; and
FIG. 2 is a schematic section of an automated analysis device comprising such a system.

FIG. 1 illustrates a system for analysing a cell suspension, such as, for example, a fixed cytological suspension.

Such a cytological suspension may originate, for example, from a screening operation involving a cervical smear, for example, using a sampling brush.

The brush is then introduced into a bottle which contains a fixing agent in order to collect the cells and thus form the cell suspension.

This cell suspension is then placed in a decantation pit, generally designated 1 in the Figure.

During the decantation operation, the cells are deposited at the bottom of this pit.

In the system according to the invention, the decantation pit is associated with means for analysing these cell deposits at the bottom of the pit over time and during the decantation operation, in order to allow the cells to be analysed by means of strata in accordance with their decantation speed.

It has been found that the pathological cells are heavier than the other cells and that they are therefore deposited first during the decantation operation.

By analysing the depositing operation of these cells, during the decantation operation and in particular the depositing of the heaviest cells, it is possible to facilitate the detection of pathological cases.

This can be carried out, for example, by means for photographing the deposits during the decantation operation, these photographing means being generally designated 2 in this Figure and being associated with means for illuminating the pit and, for example, the bottom of the pit, these illumination means being generally designated 3.

It is therefore conceivable that monitoring the decantation operation allows, for example, the analysis of the deposited cells in accordance with their weight and over time.

This allows, for example, analyses to be obtained in deposit strata, such as, for example, the strata designated T1, T2 and T3 in this FIG. 1.

An image of each deposit stratum allows the detection of pathological cells to be facilitated.

Of course, different types of analysis means may be envisaged.

These may be constituted, for example, by conventional photography and illumination means, for example, a camera, photography device or the like, or by more complex means which use, for example, means for generating wide or specific spectrum optical beams which allow specific features of the cells to be made to appear in order to detect the relevant cells or better characterise them.

It is conceivable that an analysis of the cells over time, during the decantation operation, allows each deposit stratum to be analysed and the detection of pathological cells to be facilitated.

It should also be noted that the pit, and more specifically, the open end thereof, can be associated with opaque means such as, for example, an opaque blocking membrane which is, for example, self-curing and which allows the formation, in the decantation pit, of a dark chamber which facilitates the analysis of the deposits.

Of course, other means may be envisaged for blocking the pit and forming a dark chamber.

Such an analysis system may, for example, be integrated in an automated analysis device such as that illustrated in FIG. 2 and generally designated 4.

It is possible to see, in this FIG. 2, the depositing system described with reference to FIG. 1, with the decantation pit 1, the photography means 2, the illumination means 3.

These photography and illumination means are associated, for example, with a control device for monitoring the operation of the automated device which also controls the operation of a handling arm which is generally designated 5 and suspension transfer means which are generally designated 6.

This allows, for example, the sampling means 6 to remove a sample of cell suspension from any receptacle 7, which is placed by an operator in the automated device 4, in order to transfer this sample into the decantation pit in an automated manner.

The results of the analysis are provided by the monitoring control device which is generally designated 8.

It is conceivable for such a structure to allow the analysis to be completely automated.

Of course, other embodiments may also be envisaged.

1. System for analysing a cell suspension, characterised in that it comprises at least one pit (1) for decanting the suspension and means (2, 3) for analysing the cell deposits at the bottom of the pit over time and during the decantation, in order to allow an analysis of the cells in strata (T1, T2, T3) in accordance with their decantation speed.

2. Analysis system according to claim 1, characterised in that the analysis means comprise means (3) for illuminating the pit and means (2) for photographing the deposits.

3. Analysis system according to claim 1, characterised in that the pit (1) has an open end which can be blocked by opaque means.

4. Analysis system according to claim 3, characterised in that the opaque means are formed by means of a self-curing membrane.

5. Analysis system according to claim 1, characterised in that the cell suspension is a cytological suspension.

6. Automated device for analysing a cell suspension, characterised in that it comprises an analysis system according to claim 1.

7. Automated device according to claim 6, characterised in that it further comprises means (6) for taking a sample of cell suspension from a receptacle (7) and means (5, 6) for pouring this sample into a decantation pit (1) of the analysis system.

8. Analysis system according to claim 2, characterised in that the pit (1) has an open end which can be blocked by opaque means.
